(19) United States
(12) Patent Application Publication
(10) Pub. No.: US 2006/0235001 A1
(43) Pub. Date: Oct. 19, 2006

(54) Compositions for the treatment of neoplasms

(76) Inventors: Peter Elliott, Marlborough, MA (US); Daniel Grau, Arlington, MA (US); Mahesh Padval, Waltham, MA (US); George N. Serbedzija, Sudbury, MA (US)

Correspondence Address: CLARK & ELBING LLP, 101 FEDERAL STREET, BOSTON, MA, 02110, US

(21) Appl. No.: 11/406,213

(22) Filed: Apr. 18, 2006

Related U.S. Application Data
(60) Provisional application No. 60/672,810, filed on Apr. 19, 2005.

(51) Int. Cl. A61K 31/655; A61K 31/55; A61K 31/5415; A61K 31/538; A61K 31/473; A61K 31/496

(52) U.S. Cl. 514/217; 514/225.8; 514/229.8; 514/250; 514/253.03; 514/297; 514/422; 514/150; 514/444

(57) ABSTRACT

The invention features a method for treating a patient having a cancer or other neoplasm by administering to the patient a composition that includes a phenothiazine and another active agent, where predetermined plasma drug levels are achieved and maintained for 12 hours or more.

### CROSS-REFERENCE TO RELATED APPLICATIONS

This application claims the benefit of U.S. Provisional Application No. 60/672,810, filed Apr. 19, 2005, hereby incorporated by reference.

### BACKGROUND OF THE INVENTION

The present invention relates to the treatment of neoplasms such as cancer.

Cancer is a disease marked by the uncontrolled growth of abnormal cells. Cancer cells have overcome the barriers imposed in normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

The treatment of cancer has been hampered by the fact that there is considerable heterogeneity even within one type of cancer. Some cancers, for example, have the ability to invade tissues and display an aggressive course of growth characterized by metastases. These tumors generally are associated with a poor outcome for the patient. Ultimately, tumor heterogeneity results in the phenomenon of multiple drug resistance, i.e., resistance to a wide range of structurally unrelated cytotoxic anticancer compounds, J. H. Gerlach et al., Cancer Surveys, 5:25-46 (1986). The underlying cause of progressive drug resistance may be due to a small population of drug-resistant cells within the tumor (e.g., mutant cells) at the time of diagnosis, as described, for example, by J. H. Goldie and Andrew J. Coldman, Cancer Research, 44:3643-3653 (1984). Treating such a tumor with a single drug can result in remission, where the tumor shrinks in size as a result of the killing of the predominant drug-sensitive cells. However, with the drug-sensitive cells gone, the remaining drug-resistant cells can continue to multiply and eventually dominate the cell population of the tumor. Therefore, the problems of why metastatic cancers develop pleiotropic resistance to all available therapies, and how this might be countered, are the most pressing in cancer chemotherapy.

Anticancer therapeutic approaches are needed that are reliable for a wide variety of tumor types, and particularly suitable for invasive tumors. Importantly, the treatment must be effective with minimal host toxicity. In spite of the long history of using multiple drug combinations for the treatment of cancer and, in particular, the treatment of multiple drug resistant cancer, positive results obtained using combination therapy are still frequently unpredictable. Particularly useful are those compositions that include a multiple drug combination and that are formulated to deliver to a patient a maximally effective dose over an extended period of time.

### SUMMARY OF THE INVENTION

The present invention provides anti-neoplastic compositions of phenothiazines and/or antifungal/anti-protozoal compounds, and methods for their use, where the compositions are formulated to maintain plasma levels of active components for predetermined periods of time to effectively inhibit tumor growth in a treated patient.

Accordingly, in a first aspect the invention features a method of treating a neoplasm in a human patient that includes administering a composition including a compound of formula I and/or a compound of formula II, where a first plasma level of between 0.3 ng/mL and 3.5 μg/mL for the compound of formula I and a second plasma level of between 0.2 ng/mL and 2.5 μg/mL for the compound of formula II is maintained for at least 12 hours. In one embodiment, the first plasma level is between 0.3 μg/mL and 3.5 μg/mL. In another embodiment, the second plasma level is between 0.25 μg/mL and 2.5 μg/mL. The compound of formula I has the formula: 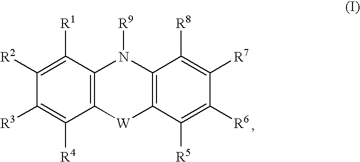
or a pharmaceutically acceptable salt or prodrug thereof, wherein

each of R¹, R³, R⁴, R⁵, R⁶, R⁷, and R⁸ is, independently, H, OH, F, OCF₃, or OCH₃;

R² is selected from the group consisting of: CF₃, halo, OCH₃, COCH₃, CN, OCF₃, COCH₂CH₃, CO(CH₂)₂CH₃, and SCH₂CH₃;

R⁹ is selected from the group consisting of: 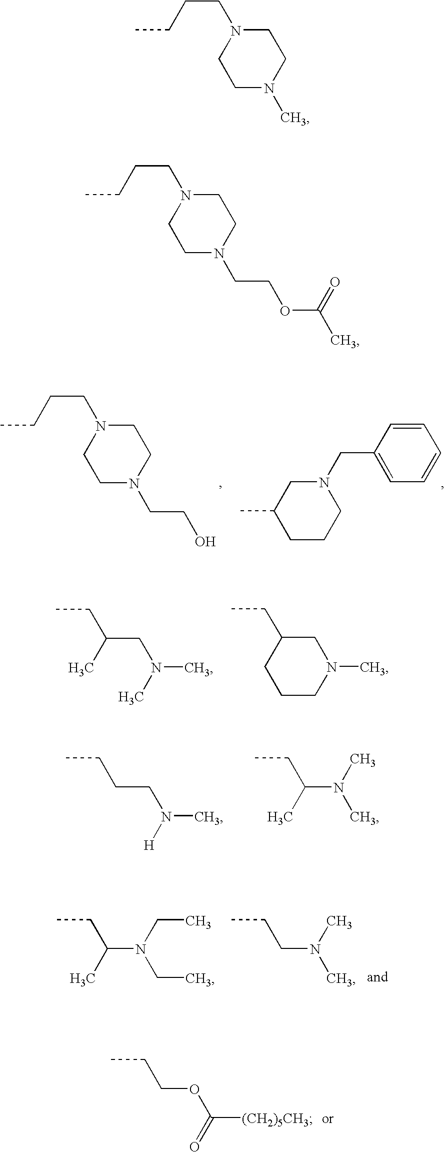
R⁹ has the formula:

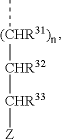

wherein n is 0 or 1, Z is NR³⁴R³⁵ or OR³⁶; each of R³¹, R³², R³³, R³⁴, R³⁵, and R³⁶ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³², R³³, R³⁴, R³⁵, and R³⁶ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; and

W is selected from the group consisting of:

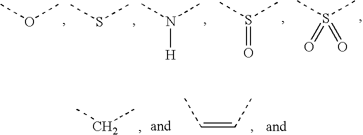

said compound of formula II is: 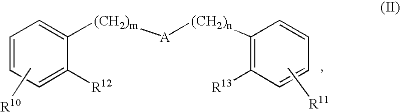
or a pharmaceutically acceptable salt or prodrug thereof, wherein

A is 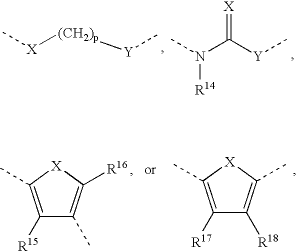
each of X and Y is, independently, O, NR¹⁹, or S, each of R¹⁴ and R¹⁹ is, independently, H or C₁₋₆ alkyl, each of R⁵, R¹⁶, R¹⁷, and R¹⁸ is, independently, H, C₁₋₆ alkyl, halogen, C₁₋₆ alkoxy, C₆₋₁₈ aryloxy, or C₆₋₁₈ aryl-C₁₋₆ alkoxy, and p is an integer of 2 to 6;

each of m and n is, independently, an integer of 0 to 2;

each of R¹⁰ and R¹¹ is 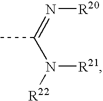
wherein R²¹ is H, C₁₋₆ alkyl, C₃₋₈ cycloalkyl, C₁₋₆ alkoxy-C₁₋₆ alkyl, hydroxy C₁₋₆ alkyl, C₁₋₆ alkylamino-C₁₋₆ alkyl, amino-C₁₋₆ alkyl, or C₆₋₁₈ aryl; R²² is H, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, carbo(C₁₋₆ alkoxy), carbo(C₆₋₁₈ aryl-C₁₋₆ alkoxy), carbo(C₆₋₈ aryloxy), or C₆-C₁₈ aryl; and R²⁰ is H, OH, or C₁₋₆ alkoxy, or R²⁰ and R²¹ together represent 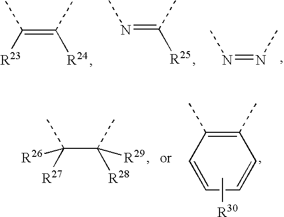
wherein each of R²³, R²⁴, and R²⁵ is, independently, H, C₁₋₆ alkyl, halogen, or trifluoromethyl, each of R²⁶, R²⁷, R²⁸, and R²⁹ is, independently, H or C₁₋₆ alkyl, and R³⁰ is H, halogen, trifluoromethyl, OCF₃, NO₂, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, or C₆₋₁₈ aryl; and

each of R¹² and R¹³ is, independently, H, Cl, Br, OH, OCH₃, OCF₃, NO₂, and NH₂, or R¹² and R¹³ together form a single bond.

The neoplasm can be, for example, selected from the group consisting of: lung cancer, colon cancer, cancer of the ovary, prostate cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, hepatocarcinoma, non-small cell lung carcinoma, multiple myeloma, mucin-depleted foci (MDF), fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Particular examples of treated neoplasms are lung cancer, colon cancer, cancer of the ovary, and prostate cancer.

Methods of treating a neoplasm by administering a composition of the invention can be performed using any formulation of the composition or method for its delivery described herein.

In another aspect, the invention features a composition that includes a compound of formula I and/or a compound of formula II, where the composition is administered to a human patient and formulated for maintaining for at least 12 hours a first plasma level of between 0.3 ng/mL and 3.5 μg/mL for the compound of formula I and/or a second plasma level of between 0.2 ng/mL and 2.5 μg/mL for the compound of formula II. Desirably, the first plasma level is between 0.3 μg/mL and 3.5 μg/mL and the second plasma level is between 0.2 μg/mL and 2.5 μg/mL. For any of the compositions of the invention that include both a compound of formula I and a compound of formula II, the weight to weight ratio of the compound of formula I to the compound of formula II can be between 1 to 10 and 10 to 1. Desirably, the weight ratio is between 1 to 2 and 1 to 5 for the compounds of formula I to formula II, respectively. Examples include ratios of about 1 to 2.5 and about 1 to 4. The compounds of formulas I and II are as defined previously herein.

For any of the compositions of the invention, desirably the compound of formula I is chlorpromazine and the compound of formula II is pentamidine.

In one embodiment, the composition is formulated for extended release. In another embodiment, the composition is formulated for continuous infusion. Predetermined first and second plasma levels can be maintained for 1 day, 2 days, 3 days, 7 days, 10 days, 14 days, 28 days, or 6 months. In order to maintain the plasma levels of a compound of formula I and/or a compound of formula II, the composition can be administered once or more than once.

In another embodiment, the growth of a neoplasm is inhibited in a human patient that obtains the predetermined plasma levels of a compound of formula I and/or a compound of formula II for a predetermined period of time. Desirably, if the composition includes a compound of formula I, the patient does not experience a substantial amount of sedation during this period.

In another aspect, the invention features a composition that includes a compound of formula I and/or a compound of formula II, where the composition is formulated for administering to a human patient by continuous intravenous infusion at a first infusion rate of between 0.1 mg/m²/hour and 15 mg/m²/hour, desirably between 1 mg/m²/hour and 5 mg/m²/hour, for the compound of formula I and at a second infusion rate of between 0.1 mg/m²/hour and 60 mg/m²/hour, desirably between 1 mg/m²/hour and 20 mg/m²/hour, for the compound of formula II. The compounds of formulas I and II are as defined previously herein.

In one embodiment, the composition is continuously infused for 12 hours, 1 day, 2 days, 3 days, 7 days, 10 days, 14 days, or 28 days. Non-limiting examples of infusion methods include the use of an intravenous drip, a peristaltic pump, or an osmotic pump.

In another embodiment, when the composition includes both a compound of formula I and a compound of formula II, the growth of a neoplasm is inhibited in a human patient that is administered the composition at predetermined first and second infusion rates for compounds of formulas I and II, respectively, for a predetermined period of time. Desirably, the patient does not experience a substantial amount of sedation during this period.

For any of the compositions of the invention, the active components can be formulated with or without excipients. Non-limiting examples of desirable excipients include between about 1 weight % and 10 weight % ascorbic acid, and between 3 weight % and 30 weight % mannitol, where each can be included with the active components alone or in various combinations with each other. Other non-limiting examples of excipients include tocopherols, cysteine, glutathione, acetone sodium bisulfite, BHA, BHT, sucrose, trehalose, sorbitol, povidone, lactose, salts of acetic acid, salts of citric acid, salts of glutamic acid, salts of phosphoric acid, dextrose, and sodium sulfate. If the composition, or a component of the composition that has been individually formulated, is a solid, it can be reconstituted with any physiologically acceptable diluent. Non-limiting examples of diluents are normal or half-normal saline and 1 weight %-10 weight % dextrose, desirably 5 weight % dextrose, where the active components constitute between about 0.005 weight % and 0.5 weight %, desirably between about 0.01 weight % and 0.2 weight %, once dissolved or suspended in the diluent. Other non-limiting examples of diluents include sterile water, Ringer's injection (NaCl+KCl+CaCl₂), lactated Ringer's injection (NaCl+KCl+CaCl₂+Na lactate), and multiple electrolyte solutions (varying combinations of electrolytes, dextrose, fructose, and/or invert sugar). In addition, diluents can also include a suitable organic co-solvent, such as, for example, ethanol or DMSO, at 0.01% to 10% of the total volume.

Any of the compositions of the invention can include one or more active agents in addition to a compound of formula I and/or a compound of formula II. For example, a composition of the invention can include an antiproliferative agent, e.g., paclitaxel, combined with a compound of formula I, e.g., chlorpromazine, and/or a compound of formula II, e.g., pentamidine.

Definitions

The term “about,” as used herein, means ±10% of the stated value.

The terms “acyl” represents an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups include from 2 to 7 carbons.

The term “alkenyl,” as used herein, represents monovalent straight or branched chain groups of, unless otherwise specified, from 2 to 6 carbons containing one or more carbon-carbon double bonds and is exemplified by ethenyl, 1-propenyl, 2-propenyl, 2-methyl-1-propenyl, 1-butenyl, 2-butenyl, and the like.

The terms “C_{x-y} alkaryl” or “C_{x-y} alkylenearyl,” as used herein, represent a chemical substituent of formula —RR′, where R is an alkyl group carbons and R′ is an aryl group as defined elsewhere herein, where x-y is the range of carbons that encompasses both groups. Similarly, by the terms “C_{x-y} alkheteroaryl” “C_{x-y} alkyleneheteroaryl,” is meant a chemical substituent of formula RR″, where R is an alkyl group of x to y carbons and R″ is a heteroaryl group as defined elsewhere herein. Other groups preceded by the prefix “alk-” or “alkylene-” are defined in the same manner.

The term “alkoxy” represents a chemical substituent of formula —OR, where R is an alkyl group of 1 to 6 carbons, unless otherwise specified.

The terms “alkyl” and the prefix “alk-,” as used herein, are inclusive of both straight chain and branched chain saturated groups of from 1 to 6 carbons, unless otherwise specified. Alkyl groups are exemplified by methyl, ethyl, n- and iso-propyl, n-, sec-, iso- and tert-butyl, neopentyl, and the like, and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy; (7) aryloyl; (8) azido; (9) carboxaldehyde; (10) cycloalkyl of three to eight carbon atoms; (11) halo; (12) heterocyclyl; (13) (heterocycle)oxy; (14) (heterocycle)oyl; (15) hydroxyl; (16) N-protected amino; (17) nitro; (18) oxo; (19) spiroalkyl of three to eight carbon atoms; (20) thioalkoxy of one to six carbon atoms; (21) thiol; (22) —C O₂R^{A}, where R^{A} is selected from the group consisting of (a) alkyl, (b) aryl and (c) alkaryl, where the alkylene group is of one to six carbon atoms; (23) —C (O)NR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (24) —SO₂R^{D}, where R^{D} is selected from the group consisting of (a) alkyl, (b) aryl and (c) alkaryl, where the alkylene group is of one to six carbon atoms; (25) —C O₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The term “alkynyl,” as used herein, represents monovalent straight or branched chain groups of from two to six carbon atoms containing a carbon-carbon triple bond and is exemplified by ethynyl, 1-propynyl, and the like.

The term “amino,” as used herein, represents an —NH₂-group.

The term “aminoalkyl,” as used herein, represents an alkyl group, as defined herein, substituted by an amino group.

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like, and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) heteroaryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where R^{B} and R^{C} are independently selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “arylalkoxy,” as used herein, represents an alkaryl group attached to the parent molecular group through an oxygen atom. Exemplary unsubstituted arylalkoxy groups are of from 7 to 16 carbons.

The term “aryloxy” represents a chemical substituent of formula —OR′, where R′ is an aryl group of 6 to 18 carbons, unless otherwise specified.

As used herein, the terms “cancer” or “neoplasm” or “neoplastic cells” represent a collection of cells multiplying in an abnormal manner. Cancer growth is uncontrolled and progressive, and occurs under conditions that would not elicit, or would cause cessation of, multiplication of normal cells.

The term “ascorbic acid,” as used herein, represents ascorbic acid, a base form of ascorbic acid, or a mixture thereof. Non-limiting examples of base forms of ascorbic acid include sodium ascorbate, potassium ascorbate, calcium ascorbate, and magnesium ascorbate. In one particular embodiment, ascorbic acid represents a 1 to 1 mixture of ascorbic acid and sodium ascorbate.

The term “cycloalkyl,” as used herein, represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H,} where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

By “formulated for extended release” is meant a formulation that, when administered to a patient, releases one or more active components from a chemical matrix over predetermined period of time. Non-limiting examples of extended release formulations include controlled release, sustained release, timed release, and delayed release formulations, as well as depot, transdermal, or mucosal formulations.

By “antiproliferative agent” is meant an agent that is capable of slowing or stopping cell proliferation, e.g., any of the agents listed in Table 1.

TABLE 1

| | | |
|---|---|---|
| Alkylating agents | Busulfan | procarbazine |
| | dacarbazine | altretamine |
| | ifosfamide | estramustine phosphate |
| | hexamethylmelamine | mechlorethamine |
| | thiotepa | streptozocin |
| | dacarbazine | temozolomide |
| | lomustine | Semustine |
| | cyclophosphamide | cisplatin |
| | chlorambucil | |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | SM-11355(Sumitomo) |
| | ZD-0473 (AnorMED) | AP-5280 (Access) |
| | oxaliplatin | |
| | carboplatin | |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | J-107088 (Merck & Co) |
| | rebeccamycin analogue (Exelixis) | BNP-1350 (BioNumerik) |
| | BBR-3576 (Novuspharma) | CKD-602 (Chong Kun Dang) |
| | rubitecan (SuperGen) | KW-2170 (Kyowa Hakko) |
| | irinotecan(CPT-11) | hydroxycamptothecin (SN-38) |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | MEN-10755 (Menarini) |
| | porfiromycin | GPX-100 (Gem Pharmaceuticals) |
| | mitoxantrone (novantrone) | Epirubicin |
| | amonafide | mitoxantrone |
| | | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B (PharmaMar) |
| | T 900607 (Tularik) | ZD 6126 (AstraZeneca) |
| | T 138067 (Tularik) | AZ10992 (Asahi) |
| | cryptophycin 52 (Eli Lilly) | IDN-5109 (Indena) |
| | vinflunine (Fabre) | AVLB (Prescient NeuroPharma) |
| | auristatin PE (Teikoku Hormone) | azaepothilone B (BMS) |
| | BMS 247550 (BMS) | BNP-7787 (BioNumerik) |
| | BMS 184476 (BMS) | CA-4 prodrug (OXiGENE) |
| | BMS 188797 (BMS) | dolastatin-10 (NIH) |
| | taxoprexin (Protarga) | CA-4 (OXiGENE) |
| | SB 408075 (GlaxoSmithKline) | docetaxel |
| | vinorelbine | vincristine |
| | | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum Pharmaceuticals) |
| | thymectacin (NewBiotics) | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenies) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenies) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | dexamethasone |
| antihormonal | conjugated estrogens | prednisone |
| agents | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; fluoxymesterone | P-04 (Novogen) |
| | methyltestosterone | 2-methoxyestradiol (EntreMed) |
| | diethylstilbestrol | arzoxifene (Eli Lilly) |
| | megestrol | tamoxifen |
| | bicalutamide | toremofine |
| | flutamide | goserelin |
| | nilutamide | Leuporelin |
| | | bicalutamide |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia ) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | | Gefitinib (Iressa) |

Miscellaneous agents

| | |
|---|---|
| SR-27897 (CCK A inhibitor, Sanofi-Synthelabo) | ceflatonin (apoptosis promotor, ChemGenex) |
| tocladesine (cyclic AMP agonist, Ribapharm) | BCX-1777 (PNP inhibitor, BioCryst) |
| alvocidib (CDK inhibitor, Aventis) | ranpirnase (ribonuclease stimulant, Alfacell) |
| CV-247 (COX-2 inhibitor, Ivy Medical) | galarubicin (RNA synthesis inhibitor, Dong-A) |
| P54 (COX-2 inhibitor, Phytopharm) | tirapazamine (reducing agent, SRI International) |
| CapCell ™ (CYP450 stimulant, Bavarian Nordic) | N-acetylcysteine (reducing agent, Zambon) |
| GCS-100 (gal3 antagonist, GlycoGenesys) | R-flurbiprofen (NF-kappaB inhibitor, Encore) |
| G17DT immunogen (gastrin inhibitor, Aphton) | 3CPA (NF-kappaB inhibitor, Active Biotech) |
| efaproxiral (oxygenator, Allos Therapeutics) | seocalcitol (vitamin D receptor agonist, Leo) |
| PI-88 (heparanase inhibitor, Progen) | 131-I-TM-601 (DNA antagonist, TransMolecular) |
| tesmilifene (histamine antagonist, YM BioSciences) | eflornithine (ODC inhibitor , ILEX Oncology) |
| histamine (histamine H2 receptor agonist, Maxim) | minodronic acid (osteoclast inhibitor, Yamanouchi) |
| tiazofurin (IMPDH inhibitor, Ribapharm) | indisulam (p53 stimulant, Eisai) |
| cilengitide (integrin antagonist, Merck KGaA) | aplidine (PPT inhibitor, PharmaMar) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug , Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| rituximab (CD20 antibody, Genentech | |

The terms “halide” or “halogen” or “halo,” as used herein, represent bromine, chlorine, iodine, or fluorine.

The terms “heterocycle” or “heterocyclyl,” as used interchangeably herein, represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocycle” also includes bicyclic, tricyclic and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring, such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

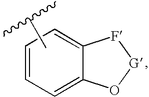

F′ is selected from the group consisting of —CH₂—, —CH₂O— and —O—, and G′ is selected from the group consisting of —C(O)— and —(C(R′)(R″))ᵥ—, where each of R′ and R″ is, independently, selected from the group consisting of hydrogen or alkyl of one to four carbon atoms, and v is one to three and includes groups, such as 1,3-benzodioxolyl, 1,4-benzodioxanyl, and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) heteroaryl; (14) alkaryl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) alkaryl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) alkaryl, where the alkylene group is of one to six carbon atoms; (38) —(CH₂)_{q}SO₂R^{D}, where R^{D} is selected from the group consisting of (a) alkyl, (b) aryl and (c) alkaryl, where the alkylene group is of one to six carbon atoms; (39) —(CH₂)_{q}SO₂NR^{E}R^{F}, where each of R^{E} and R^{F} is, independently, selected from the group consisting of (a) hydrogen, (b) alkyl, (c) aryl and (d) alkaryl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) alkaryl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “hydroxy,” as used herein, represents an —OH group.

By “inhibits the growth of a neoplasm” is meant measurably slows, stops, or reverses the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “infusion rate” is meant a rate of infusion of an active agent, such as, for example, a compound of formula I or II, from a composition that does not vary positively or negatively by more than 25% from the mean rate of infusion of the active agent over a prolonged period of time, such as, for example, 12 hours or more.

By “normal saline” is meant a solution of sodium chloride that has the same electrolytic balance as found in serum. A normal saline solution is 0.9 weight percent sodium chloride in water. A half-normal saline solution is 0.45 weight percent sodium chloride in water.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable prodrugs,” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “prodrug,” as used herein, represents compounds which are rapidly transformed in vivo to the parent compound of the above formula, for example, by hydrolysis in blood. Prodrugs of the compounds of the invention may be conventional esters. Some common esters which have been utilized as prodrugs are phenyl esters, aliphatic (C₈-C₂₄) esters, acyloxymethyl esters, carbamates and amino acid esters. For example, a compound of the invention that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

By “substantial degree of sedation” is meant that amount of sedation that prevents a treated subject from performing normal activities, such as, for example, walking, conversing, and eating.

### BRIEF DESCRIPTION OF THE DRAWINGS

FIG. 1 is a graph comparing the serum plasma concentrations of the components of a chlorpromazine/pentamidine composition that were observed when the composition was administered by continuous infusion vs. intraperitoneal bolus injection in a mouse tumor model study.

FIG. 2 is a graph demonstrating tumor growth inhibition when a chlorpromazine/pentamidine composition was administered by either bolus intraperitoneal injection or continuous infusion in the mouse tumor model study.

FIG. 3 is a graph showing reduced weight loss in the mice treated with a continuously infused chlorpromazine/pentamidine composition vs. those treated with an i.p. bolus injection of a chlorpromazine/pentamidine composition.

### DETAILED DESCRIPTION

Synergistic combinations of phenothiazines and antifungal/anti-protozoal agents have been described that effectively inhibit tumor growth (see U.S. Patent Application Publication No. 20040116407). The present invention features compositions of phenothiazines of formula I and/or antifungal/anti-protozoal compounds of formula II, where the compositions are formulated to maintain plasma levels of these compounds such that tumor growth is effectively inhibited in a treated patient. In addition to potentially reducing the sedation side effects that correspond to the administration of phenothiazines in those compostions that contain them, such compositions have an improved safety profile.

Accordingly, in a first aspect the invention features a composition formulated to maintain for at least 12 hours in a treated patient a plasma level between 0.3 ng/mL (about 1.0 nanomolar) and 3.5 μg/mL (about 10 micromolar) of a compound of formula I and/or between 0.2 ng/mL (about 1.0 nanomolar) and 2.5 μg/mL (about 10 micromolar) of a compound of formula II, where the compound of formula I is: 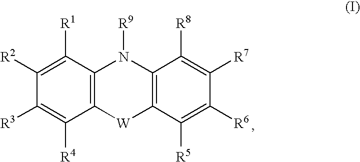
or a pharmaceutically acceptable salt or prodrug thereof, wherein

each of R¹, R³, R⁴, R⁵, R⁶, R⁷, and R⁸ is, independently, H, OH, F, OCF₃, or OCH₃;

R² is selected from the group consisting of: CF₃, halo, OCH₃, COCH₃, CN, OCF₃, COCH₂CH₃, CO(CH₂)₂CH₃, and SCH₂CH₃;

R⁹ is selected from the group consisting of:

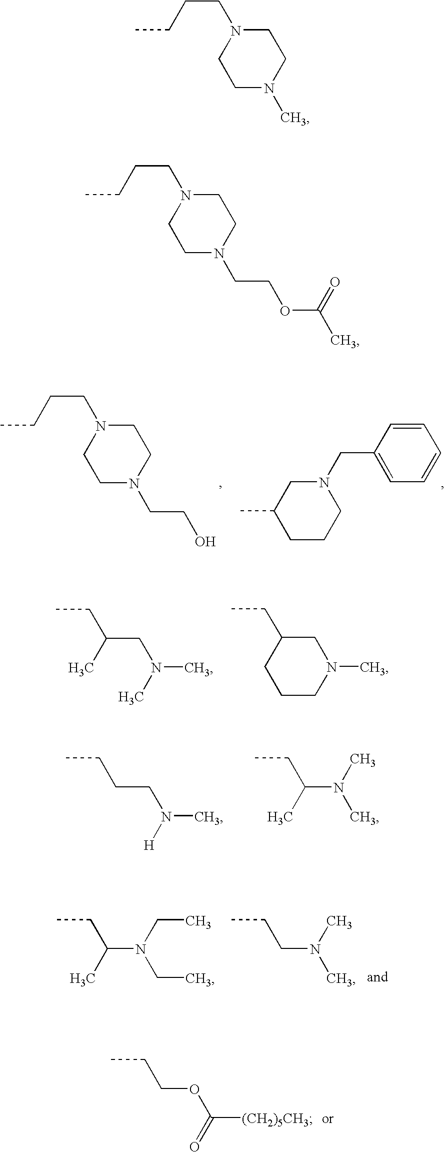

R⁹ has the formula:

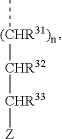

wherein n is 0 or 1, Z is NR³⁴R³⁵ or OR³⁶; each of R³¹, R³², R³³, R³⁴, R³⁵, and R³⁶ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³², R³³, R³⁴, R^{35,} and R³⁶ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; and

W is selected from the group consisting of:

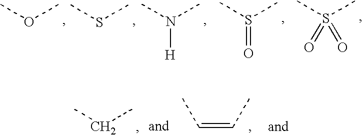

said compound of formula II is: 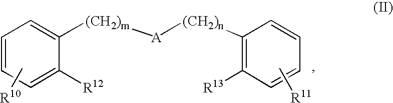
or a pharmaceutically acceptable salt or prodrug thereof, wherein

A is 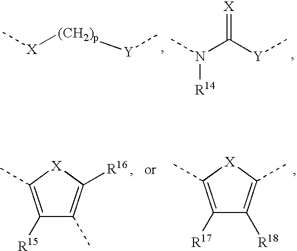
each of X and Y is, independently, O, NR¹⁹, or S, each of R¹⁴ and R¹⁹ is, independently, H or C₁₋₆ alkyl, each of R¹⁵, R¹⁶, R¹⁷, and R¹⁸ is, independently, H, C₁₋₆ alkyl, halogen, C₁₋₆ alkoxy, C₆₋₁₈ aryloxy, or C₆₋₁₈ aryl-C₁₋₆ alkoxy, and p is an integer of 2 to 6;

each of m and n is, independently, an integer of 0 to 2;

each of R¹⁰ and R¹¹ is 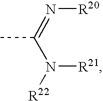
wherein R²¹ is H, C₁₋₆ alkyl, C₃₋₈ cycloalkyl, C₁₋₆ alkoxy-C₁₋₆ alkyl, hydroxy C₁₋₆ alkyl, C₁₋₆ alkylamino-C₁₋₆ alkyl, amino-C₁₋₆ alkyl, or C₆₋₁₈ aryl; R²² is H, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, carbo(C₁₋₆ alkoxy), carbo(C₆₋₁₈ aryl-C₁₋₆ alkoxy), carbo(C₆₋₁₈ aryloxy), or C₆-C₁₈ aryl; and R²⁰ is H, OH, or C₁₋₆ alkoxy, or R²⁰ and R²¹ together represent 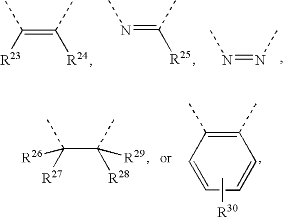
wherein each of R²³, R²⁴, and R²⁵ is, independently, H, C₁₋₆ alkyl, halogen, or trifluoromethyl, each of R²⁶, R²⁷, R²⁸, and R²⁹ is, independently, H or C₁₋₆ alkyl, and R³⁰ is H, halogen, trifluoromethyl, OCF₃, NO₂, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, or C₆₋₁₈ aryl; and

each of R¹² and R¹³ is, independently, H, Cl, Br, OH, OCH₃, OCF₃, NO₂, and NH₂, or R¹² and R¹³ together form a single bond.

In one embodiment, the compound of formula I is chlorpromazine and/or the compound of formula II is pentamidine.

In another embodiment, the plasma level of the compound of formula I is between 0.3 μg/mL and 3.5 μg/mL and/or the plasma level of the compound of formula II is between 0.2 μg/mL and 2.5 μg/mL. In another embodiment, the plasma level of the compound of formula I is between 10 ng/mL and 1 μg/mL and/or the plasma level of the compound of formula II is between 10 ng/mL and 1 μg/mL. In yet another embodiment, the plasma level of the compound of formula I is between 0.5 μg/mL and 3.5 μg/mL and/or the plasma level of the compound of formula II is between 0.5 μg/mL and 2.5 μg/mL. Plasma levels can be maintained for 12 hours or more, 24 hours or more, 3 days or more, 7 days or more, 14 days or more, 28 days or more, or 6 months or more.

Formulations

The compositions of the invention are formulated for delivery to a human patient such that the plasma levels of the active components are maintained at predetermined levels for a predetermined period of time. Methods in the art are known for achieving extended release according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

A particular composition of the invention includes chlorpromazine as the compound of formula I and pentamidine as the compound of formula II. Non-limiting examples of suitable weight/weight pentamidine/chlorpromazine ratios range from 2 to 1 to 4 to 1, and include pentamidine/chlorpromazine ratios of 2 to 1, 2.25 to 1, 2.5 to 1, 2.75 to 1, 3 to 1, 3.25 to 1, 3.5 to 1, 3.75 to 1, and 4to 1. The composition components can be formulated separately or together. In one example, the composition components are formulated together as a lyophilized powder. In another example, the composition components are formulated separately, reconstituted, and combined.

Suitable excipients are known to those skilled in the art and are described in Remington, vide supra. In one example, the composition includes ascorbic acid (each between 1 weight % and 10 weight %, desirably between 2 weight % and 4 weight %). In another example, the composition includes mannitol (between 3 weight % and 30 weight %). In yet another example, the composition includes ascorbic acid and mannitol, with the composition including each of these excipients within the weight % range previously defined.

Solid formulations can be reconstituted in a suitable liquid, such as, for example, a 1 weight %-10 weight % dextrose solution or saline at half-normal or normal concentrations, to form a composition of the invention, where each of the active components of the composition (i.e., the compounds of formula I and II) has a final concentration between about 0.005 weight % and 0.5 weight %.

Table 2 provides several non-limiting examples of components of a composition of the invention, where the composition includes chlorpromazine and pentamidine, and optionally includes ascorbic acid and/or mannitol as excipients. Each formulation is dissolved in about 100 mL to 500 mL of normal saline or 5 weight % dextrose to form the composition.

TABLE 2

| | Amount of Ingredient Contained in One Vial | | | | |
|---|---|---|---|---|---|
| | Chlorpromazine | Pentamidine | Sodium | Ascorbic | |
| Number | Hydrochloride | Isethionate | Ascorbate | Acid | Mannitol |
| F1 | 50 mg/vial | 120 mg/vial | 2.5 mg/vial | 2.5 mg/vial | 43.7 mg/vial |
| F3 | 50 mg/vial | 120 mg/vial | 2.5 mg/vial | 2.5 mg/vial | |
| F5 | 50 mg/vial | 120 mg/vial | | | 55 mg/vial |
| F6 | 50 mg/vial | 200 mg/vial | | | 21 mg/vial |
| F7 | 50 mg/vial | 120 mg/vial | | | |
| F8 | 50 mg/vial | 200 mg/vial | | | |

Administration

The administration of the compositions of the invention may be by any suitable means that results in a concentration of the active agents that is anti-neoplastic upon reaching the target region. The compound may be contained in any appropriate amount in any suitable carrier substance. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Desirably, the composition is formulated for extended release, which can be achieved by a variety of methods. Two common methods include: 1) providing an extended release coating upon tablets or microspheres wherein slow release of the active ingredient occurs via either gradual permeation through or gradual breakdown of this coating; and 2) providing an extended release matrix, such as a fat, a wax, or a polymeric material intermixed with the active ingredient in the tablet itself. These are described, for example, in The Theory and Practice of Industrial Pharmacy, Manford Robinson, Chapter 14, “Sustained Action Dosage Forms,” L. Lachman et al., eds., Published by Lea & Febiger, Second Ed., 1976.

Examples of the former method include the use of osmotic devices, which are known for their ability to provide a controlled release of a wide range of drugs. Known devices include tablets, pastilles, pills or capsules and others and generally include layers comprising one or more materials that are subject to erosion or that slowly dissolve in the environment of use thereby gradually dispensing the active agent. U.S. Pat. No. 4,014,334 describes an osmotic device for the controlled and continuous delivery of a drug wherein the device comprises: a) a core containing a drug and an osmotic agent; b) a semipermeable laminate, surrounding the core, which includes an external semipermeable lamina and an internal semipermeable lamina; and c) a passageway which communicates the core with the exterior of the device. The two semipermeable laminae maintain their chemical and physical integrity in the presence of the drug and fluid from the environment. The passageway includes an aperture, orifice or bore through the laminate formed by mechanical procedures, or by eroding an erodible element, such as a gelatin plug, in the environment of use. Other similar osmotic devices are described in U.S. Pat. Nos. 3,845,770; 4,576,604 and 4,673,405. U.S. Pat. No. 5,558,879 describes a controlled release tablet for water soluble drugs in which a passageway is formed in the environment of use, i.e., the GI tract of a person receiving the formulation. Specifically, the controlled release tablet consists essentially of: a) a core containing a drug, 5-20% by weight of a water soluble osmotic agent, a water soluble polymer binder and a pharmaceutical carrier; and b) a dual layer membrane coating around the core consisting essentially of: (1) an inner extended-release coating containing a plasticized water insoluble polymer and a water soluble polymer; and (2) an outer immediate release coating containing a drug and a water soluble polymer. U.S. Pat. No. 4,810,502 describes an osmotic dosage form for delivering pseudoephedrine (Ps) and brompheniramine (Br) which comprises: a) a core containing Ps and Br; b) a wall surrounding the core comprising cellulose acylate and hydroxypropylcellulose; c) a passageway in the wall for delivering the drug; and d) a lamina on the outside of the wall comprising Ps, Br, at least one of hydroxypropylcellulose and hydroxypropyl methylcellulose, and poly(ethylene oxide) for enhancing the mechanical integrity and pharmacokinetics of the wall. U.S. Pat. No. 4,801,461 also describes an osmotic dosage form for delivering pseudoephedrine (Ps). Specifically, the osmotic dosage form includes: a) a core containing varying amounts of Ps; b) a semipermeable wall surrounding the core comprising varying amounts of cellulose acetate or cellulose triacetate and varying amounts of hydroxypropylcellulose; c) a passageway in the wall for delivering the drug from the core; and optionally d) a lamina on the outside of the wall comprising Ps. The core can also contain one or more of sodium chloride, microcrystalline cellulose, hydroxypropyl methylcellulose, magnesium stearate, and poly(vinylpyrrolidone). The passageway of this device can extend through the semipermeable wall alone or through both the semipermeable wall and the outer lamina. The passageway also includes materials that erode or leach in the environment of use. U.S. Pat. No. 5,681,584 describes a controlled release drug delivery device that includes: a) a core containing a drug, an optional osmotic agent and optional excipients; b) a delayed release jacket comprising at least one of a binder, an osmotic agent and a lubricant surrounding the core; c) a semipermeable membrane surrounding the delayed release jacket and optionally having a passageway; d) a drug-containing layer either on the outside of the semipermeable membrane or between the semipermeable membrane and the delayed release jacket; and e) an optional enteric coat either on the outside of the drug-containing layer, between the drug-containing layer and the semipermeable membrane or on the outside of the semipermeable membrane when the drug-containing layer is between the delayed release jacket and the semipermeable membrane. U.S. Pat. No. 6,004,582 discloses osmotic devices similar to those described above with a water soluble poly(vinylpyrrolidone)-(vinyl acetate) copolymer polymer coat between the semipermeable membrane and the drug-containing layer.

Examples of extended-release matrix formulations useful for a composition of the present invention include those disclosed in U.S. Pat. No. 4,259,314, which describes a mixture of cellulose ethers-hydroxypropylmethylcellulose (“HPMC”) and hydroxypropyl cellulose, to form a extended release matrix in which the cellulose ether mixture has a weighted average viscosity rating of 250-4500; U.S. Pat. No. 5,451,409, which describes a dry mixed tablet in which a mixture of hydroxypropyl cellulose and hydroxyethyl cellulose forms the extended-release matrix, where 0.5-10% HPMC is also added as a binder; and U.S. Pat. Nos. 4,369,172; 4,389,393, and 4,983,396, each of which describes the use of HPMC in a variety of extended-release formulations.

Additional examples of useful extended-release formulations include those disclosed in U.S. Pat. No. 6,586,005, which describes an extended-release formulation of etodolac for once daily administration; U.S. Pat. Nos. 6,509,037 and 6,312,724, which describe an extended-release formulation of diclofenac for once daily administration; and U.S. Pat. No. 6,372,252, which describes an extended-release formulation of guaifenesin for twice daily administration. Still other useful extended-release formulations are described in U.S. Pat. Nos. 3,916,899, 3,536,809, 3,598,123, 4,008,719, 4,710,384, 5,674,533, 5,059,595, 5,591,767, 5,120,548, 5,073,543, 5,639,476, 5,354,556, and 5,733,566.

For transdermal formulations, a permeation enhancer, for example, a glycolipid, a non-esterified fatty acid, an aliphatic alcohol, a fatty acid ester of an aliphatic alcohol, a cyclohexanol, a fatty acid, ester of glycerol, a glycol, or an aliphatic alcohol ether can be used. Other components such as a stabilizer, a solubilizer, a surfactant and a plasticizer can be present in a transdermal device (see, for example, U.S. Patent Application No. 20020127254).

Polymers have been used as carriers of therapeutic agents to affect extended release. See, for example, Leong et al., “Polymeric Controlled Drug Delivery”, Advanced Drug Delivery Rev. 1:199-233, 1987; Langer, “New Methods of Drug Delivery”, Science 249:1527-33, 1990; and Chien et al., Novel Drug Delivery Systems, 1982. Such delivery systems offer the potential of enhanced therapeutic efficacy and reduced overall toxicity. Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists”, Controlled Release of Bioactive Materials, 19-44, Richard Baker ed., 1980); poly(amino acids) and pseudo-poly(amino acids) (Pulapura et al. “Trends in the Development of Bioresorbable Polymers for Medical Applications”, J. Biomaterials Appl. 6(3):216-50, 1992); polyurethanes (Bruin et al., “Biodegradable Lysine Diisocyanate-based Poly(Glycolide-co-.epsilon. Caprolactone)-Urethane Network in Artificial Skin”, Biomaterials 11(4):291-95, 1990); polyorthoesters (Heller et al., “Release of Norethindrone from Poly(Ortho Esters)”, Polymer Engineering Sci. 21(11):727-31, 1981); and polyanhydrides (Leong et al., “Polyanhydrides for Controlled Release of Bioactive Agents”, Biomaterials 7(5):364-71, 1986).

Biodegradable block polymers that are suitable for drug delivery for a composition of the invention and methods of their preparation are described by Kumar et al., Adv. Drug Deliv. Rev. 53:23-44, 2001. Copolymers can be random, alternating, or block (di or tri type) and can be linear, or star or graft (comb-shaped) in configuration. A polymer can form a hydrogel, which is a three-dimensional, hydrophilic polymeric network that holds a large amount of aqueous fluid. The polymer used in a hydrogel is rendered insoluble by cross-linking or other chemical adducts.

Microparticles can be formed of polymeric microspheres that encapsulate a composition of the invention. Polymers for use in microspheres include poly(lactic acid) or PLA; poly(glycolic acid) or PGA; and copolymer PLA-PGA. Amounts of the active agents of a composition of the present invention are released in stages such as an initial burst of agents non-specifically associated with the exterior of the particles, a later stage by diffusion, and a final stage by erosion can be controlled by polymer composition, molecular weight, size of the microparticles, and physiological conditions such as pH. Microspheres can be produced from a supercritical fluid, e.g., supercritical carbon dioxide (scCO₂).

Biodegradable implants can be prepared from materials such as at least one of the materials selected from the group of: starch; vinylstarch; dipropyleneglycol diacrylate (DPGDA); tripropyleneglycol diacrylate (TPGDA); pectin; cellulose acetate; cellulose propionate; cellulose acetate butyrate; cellulose acetate propionate (CAP); hydroxypropyl cellulose (HPC); hydroxypropyl cellulose/cellulose acetate propionate (HPC/CAP); methyl methacrylate (MMA); butyl methacrylate (BMA); hydroxymethyl methacrylate (HEMA); ethyl hexyl acrylate (EHA); octadecyl methacrylate (ODMA); and ethyleneglycol dimethacrylate (EGDMA). See Gil et al., Boletim de Biotecnologia 72:13-19, 2002.

In addition to polymers, naturally occurring and synthetic lipids can be used for extended-release formulations. DepoFoam™ (Skye Pharma, London, England) forms a multivesicular lipid-based particle (liposome) for encapsulating therapeutic agents (see U.S. Pat. No. 5,993,850; and Ye et al., J. Controlled Rel. 64:155-166, 2000). The lipids are amphipathic with a net negative charge, sterols, or zwitterionic lipids, and methods for making the liposomes are non-acidic.

Other lipids can also be used for liposomes of extended-release formulations. A plant polar lipid, such as, for example, a wheat ceramide, is useful for forming a gel with a protein such as a prolamine, into which one or more therapeutic agents can be placed for transdermal or transmucosal delivery. (see, for example, U.S. Pat. No. 6,410,048). Exemplary prolamines include wheat gliadin, and corn zein. Other naturally occurring polymers used in extended-release drug formulations and devices include collagen (EP-A-0 621 044), chitin (U.S. Pat. No. 4,393,373), and chitosan, which is a deacylated form of chitin.

Lipids and a variety of types of polymers can also be used to form “nanoparticles” for the delivery of a composition of the invention (see Kumar J. Pharm. Pharmaceut. Sci. 3:234-258, 2002).

Any of the compositions of the invention can be formulated for delivery by a mechanical device to deliver the formulation over an extended period of time. The device can be, for example, a degradable implant; a transcutaneous patch; a catheter; an implantable pump; a percutaneous pump; an infusion pump; or an iontophoresis device. Mechanical delivery devices can be used alone or in combination with a formulation for controlled, sustained, timed, delayed, or extended release.

Infusion pumps are also well-known to those skilled in the art (see, for example, Burtles, “Continuous Infusion Of Drugs: A Simple And Rational System,” Journal of Cardiothoracic and Vascular Anesthesia 5(4):362-364, 1991, and Tilden and Hopkins, “Calculation Of Infusion Rates Of Vasoactive Substances,” Annals of Emergency Medicine 12:697-99, 1983).

Other pumps, which can be implantable or non-implantable (external), for delivery of a composition of the invention include peristaltic pumps, fluorocarbon propellant pumps, or osmotic pumps, including mini-osmotic pumps. Peristaltic pumps deliver a set amount of composition with each electric pulse that drives the pump head. The pump, electronics and power source are located in a titanium housing covered in Silastic. Composition reservoirs are silicone rubber pouches that can withstand substantial pressure, for example, 60 psi. The reservoir can be refilled percutaneously through a polypropylene port. Fluorocarbon pumps use a fluorocarbon liquid to operate the pump. Osmotic pumps use osmotic pressure to release the drug at a constant rate. An exemplary pump is the MiniMed MicroMed 407C pump (Medtronic, Inc., Northridge, Calif.). Further, an intrathecal drug delivery system (Medronic) which includes two implantable components, an infusion pump, and an intraspinal catheter, can be used. The pump is inserted abdominally in a subcutaneous pocket, while the catheter is inserted into the intrathecal space of the spine, tunneled under the skin, and connected to the pump. A composition of the invention can then be delivered at a constant or variable flow rate.

### EXAMPLE

An animal experiment was carried out using male 6-8 week old SCID Hsd:ICR9 CD-1 mice (Harlan, Indianapolis, Ind.). Approximately 1×10⁶ HCT116 or A549 human tumor cells (obtained directly from ATCC) were injected subcutaneously into the left and right flank of each animal. The animals were monitored for tumor growth and, once the total tumor volume reached approximately 500 mm³, animals were randomized into treatment groups (n=10).

A composition of 1:2 weight ratio of chlorpromazine/pentamidine (CRx-026) at a concentration of 0.97 mg/mL and 1.87 mg/mL, respectively, in 5% dextrose was administered for a two-week period as an i.p. bolus or at a concentration of 23 mg/mL and 44 mg/mL, respectively, in 5% dextrose solution containing 10% ethanol via an i.p. implanted ALZET osmotic mini-pump. The amount administered by either method was 5 mg chlorpromazine/kg body weight and 10 mg pentamidine/kg body weight.

Tumors were measured with calipers three times per week during the treatment period. Tumor volume was calculated using the following equation: (length×(width)²/2). Blood and tumor tissues were obtained 30 min to 24 hours post dose. The concentrations of CRx-026 components were determined by HPLC-MS-MS. Noncompartmental analysis was performed using WinNonlin 4.1.

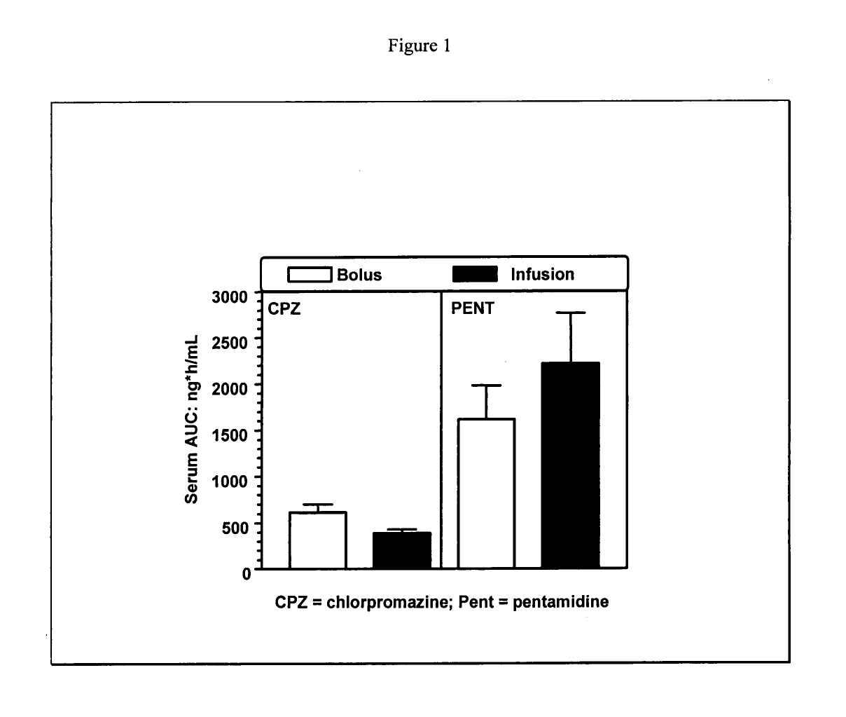

As shown in FIG. 1, continuous infusion of the chlorpromazine/pentamidine composition resulted in a systemic serum exposure that was comparable to that observed upon bolus i.v. administration. Pharmacokinetic parameters of the continuously infused composition are shown in Table 3.

TABLE 3

| | Serum Steady State | | Tumor Steady State | |
|---|---|---|---|---|
| | (ng/mL) | | (ng/g) | |
| Component | Mean | SD | Mean | SD |
| Chlorpromazine | 13.4 | 3.9 | 95.5 | 41.2 |
| Pentamidine | 83.7 | 25.2 | 848.2 | 443.2 |

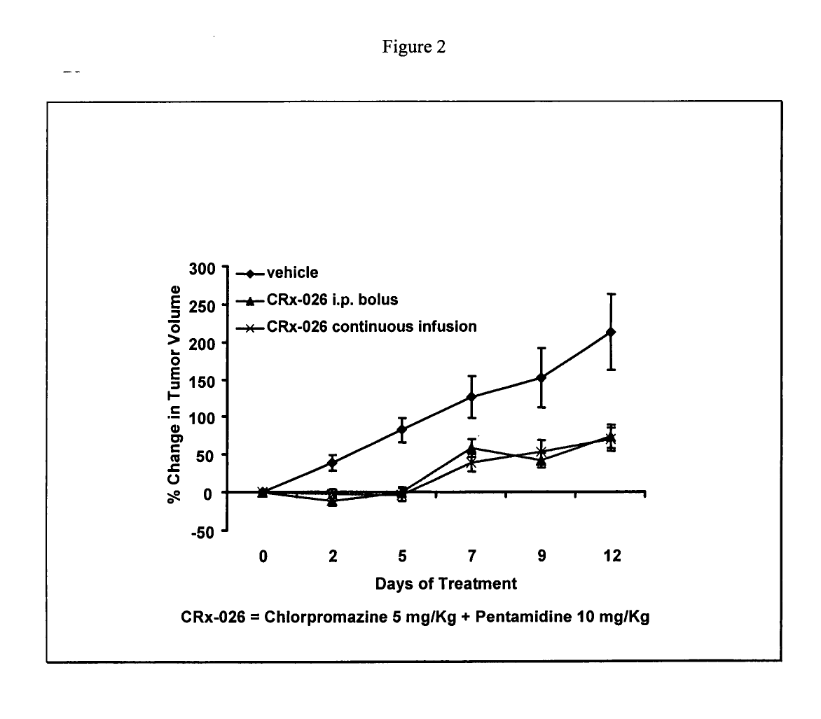

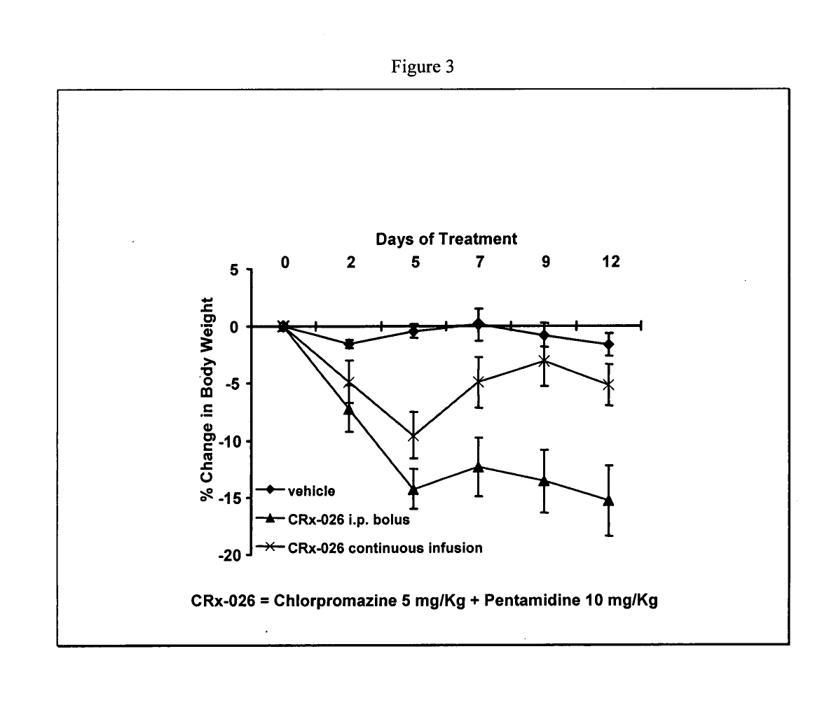

As shown in FIG. 2, the continuously infused composition resulted in a 58% decrease in tumor volume after twelve days compared to vehicle treated animals (controls), an effect that was comparable to the reduced tumor growth observed with daily i.p. administration of the composition. In addition, as shown in FIG. 3, administering the composition by continuous infusion resulted in an improved safety profile, as evidenced by a reduction in the loss of body weight of treated animals when compared to those animals subjected to a bolus injection of the composition.

### Other Embodiments

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating a neoplasm in a human patient, said method comprising administering a composition comprising a compound of formula I and a compound of formula II, wherein a first plasma level of between 0.3 ng/mL and 3.5 μg/mL for said compound of formula I and a second plasma level of between 0.2 ng/mL and 2.5 μg/mL for said compound of formula II is maintained for at least 12 hours, wherein said compound of formula I is: 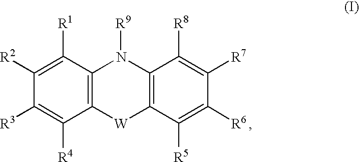
or a pharmaceutically acceptable salt or prodrug thereof, wherein
each of R¹, R³, R⁴, R⁵, R⁶, R⁷, and R⁸ is, independently, H, OH, F, OCF₃, or OCH₃;
R² is selected from the group consisting of: CF₃, halo, OCH₃, COCH₃, CN, OCF₃, COCH₂CH₃, CO(CH₂)₂CH₃, and SCH₂CH₃;
R⁹ is selected from the group consisting of: 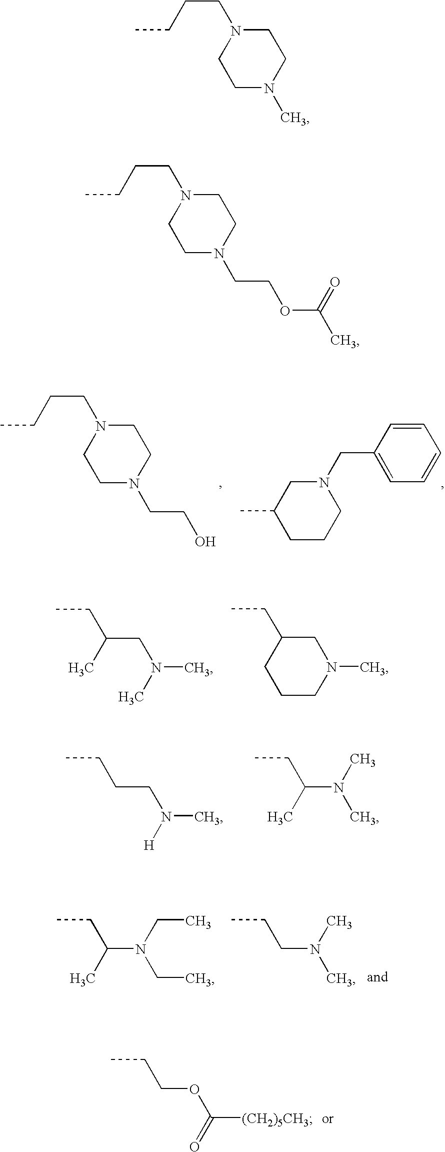
R⁹ has the formula: 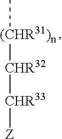
wherein n is 0 or 1, Z is NR³⁴R³⁵ or OR³⁶; each of R³¹, R³², R³³, R³⁴ R³⁵, and R³⁶ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³², R³³, R³⁴, R³⁵, and R³⁶ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; and
W is selected from the group consisting of: 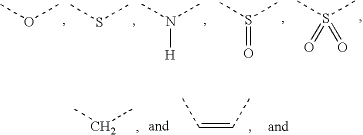
said compound of formula II is: 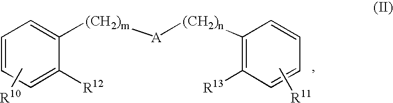
or a pharmaceutically acceptable salt or prodrug thereof, wherein
A is 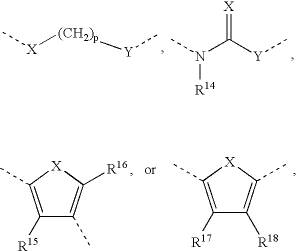
wherein each of X and Y is, independently, O, NR¹⁹, or S, each of R¹⁴ and R¹⁹ is, independently, H or C₁₋₆ alkyl, each of R¹⁵, R¹⁶, R¹⁷, and R¹⁸ is, independently, H, C₁₋₆ alkyl, halogen, C₁₋₆ alkoxy, C₆₋₁₈ aryloxy, or C₆₋₁₈ aryl-C₁₋₆ alkoxy, and p is an integer of 2 to 6;
each of m and n is, independently, an integer of 0 to 2;
each of R¹⁰ and R¹¹ is 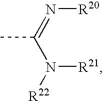
wherein R²¹ is H, C₁₋₆ alkyl, C₃₋₈ cycloalkyl, C₁₋₆ alkoxy-C₁₋₆ alkyl, hydroxy C₁₋₆ alkyl, C₁₋₆ alkylamino-C₁₋₆ alkyl, amino-C₁₋₆ alkyl, or C₆₋₁₈ aryl; R²² is H, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, carbo(C₁₋₆ alkoxy), carbo(C₆₋₁₈ aryl-C₁₋₆ alkoxy), carbo(C₆₋₁₈ aryloxy), or C₆-C₁₈ aryl; and R²⁰ is H, OH, or C₁₋₆ alkoxy, or R²⁰ and R²¹ together represent 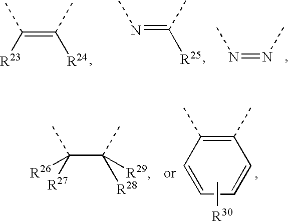
wherein each of R²³, R²⁴, and R²⁵ is, independently, H, C₁₋₆ alkyl, halogen, or trifluoromethyl, each of R²⁶, R²⁷, R²⁸, and R²⁹ is, independently, H or C₁₋₆ alkyl, and R³⁰ is H, halogen, trifluoromethyl, OCF₃, NO₂, C₁₋₆ alkyl, C₃₋₈ cycloalkyl, C₁₋₆ alkoxy, C₁₋₆ alkoxy-C₁₋₆ alkyl, hydroxyl-C₁₋₆ alkyl, C₁₋₆ alkylamino-C₁₋₆ alkyl, amino-C₁₋₆ alkyl, or C₆₋₁₈ aryl; and
each of R¹² and R¹³ is, independently, H, Cl, Br, OH, OCH₃, OCF₃, NO₂, and NH₂, or R¹² and R¹³ together form a single bond.

2. The method of claim 1, wherein said first plasma level is maintained between 0.3 μg/mL and 3.5 μg/mL and said second plasma level is maintained between 0.25 μg/mL and 2.5 μg/mL.

3. The method of claim 1, wherein the combination of said first and second plasma levels effectively inhibits the growth of a neoplasm in said patient.

4. The method of claim 3, wherein the combination of said first and second plasma levels does not induce a substantial amount of sedation in said patient.

5. The method of claim 1, wherein said compound of formula I is chlorpromazine and said compound of formula II is pentamidine.

6. The method of claim 1, wherein said composition is administered by continuous intravenous infusion at a first infusion rate of between 0.1 mg/m²/hour and 15 mg/m²/hour for said compound of formula I and at a second infusion rate of between 0.1 mg/m²/hour and 60 mg/m²/hour for said compound of formula II.

7. The method of claim 6, wherein the combination of said first and second infusion rates effectively inhibits the growth of a neoplasm in said patient.

8. The method of claim 7, wherein the combination of said first and second infusion rates does not induce a substantial amount of sedation in said patient.

9. A method of treating a neoplasm in a human patient, said method comprising administering a composition comprising a compound of formula I, wherein a plasma level of between 0.3 ng/mL and 3.5 μg/mL for said compound of formula I is maintained for at least 12 hours.

10. The method of claim 9, wherein said plasma level is between 0.3 μg/mL and 3.5 μg/mL.

11. The method of claim 9, wherein said compound of formula I is chlorpromazine.

12. A method of treating a neoplasm in a human patient, said method comprising administering a composition comprising a compound of formula II, wherein a plasma level of between 0.2 ng/mL and 2.5 μg/mL for said compound of formula II is maintained for at least 12 hours.

13. The method of claim 12, wherein said plasma level is between 0.25 μg/mL and 2.5 μg/mL.

14. The method of claim 12, wherein said compound of formula II is pentamidine.

15. The method of claim 1, 9, or 12, wherein said composition is formulated for extended release.

16. The method of claim 1, 9, or 12, wherein said neoplasm is selected from the group consisting of: lung cancer, colon cancer, cancer of the ovary, prostate cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, hepatocarcinoma, non-small cell lung carcinoma, multiple myeloma, mucin-depleted foci (MDF), fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

17. The method of claim 1, 9, or 12, wherein said neoplasm is selected from the group consisting of: lung cancer, colon cancer, cancer of the ovary, and prostate cancer.

18. The method of claim 1, 9, or 12, wherein said composition is administered by continuous intravenous infusion for at least 12 hours.

19. The method of claim 1, 9, or 12, wherein said composition is administered by continuous intravenous infusion for at least 3 days.

20. The method of claim 1, 9, or 12, wherein said composition is administered by continuous intravenous infusion for at least 7 days.

21. The method of claim 1, 9, or 12, wherein said composition is administered by an osmotic or peristaltic pump.

22. The method of claim 1, 9, or 12, wherein said composition is administered by intravenous drip.

23. The method of claim 1, 9, or 12, wherein said composition further comprises ascorbic acid.

24. The method of claim 23, wherein said ascorbic acid is between about 1 weight % and about 10 weight %.

25. The method of claim 1, 9, or 12, wherein said composition further comprises mannitol.

26. The method of claim 25, wherein said mannitol is between about 3 weight % and about 30 weight %.

27. The method of claim 1, 9, or 12, wherein said composition further comprises an antiproliferative agent of Table 1.
